(19) United States
(12) Patent Application Publication
(10) Pub. No.: US 2024/0197334 A1
(43) Pub. Date: Jun. 20, 2024

(54) EMBOLIC COIL

(71) Applicant: TERUMO KABUSHIKI KAISHA, Tokyo (JP)

(72) Inventor: Ryo OKAMURA, Fujinomiya, Shizuoka (JP)

(73) Assignee: TERUMO KABUSHIKI KAISHA, Tokyo (JP)

(21) Appl. No.: 18/593,010

(22) Filed: Mar. 1, 2024

Related U.S. Application Data
(63) Continuation of application No. PCT/JP2022/022855, filed on Jun. 6, 2022.

(30) Foreign Application Priority Data
Sep. 7, 2021 (JP) 2021-145724

(51) Int. Cl. A61B 17/12; A61B 17/00

(52) U.S. Cl. CPC A61B 17/1215; A61B 17/12031; A61B 17/12113; A61B 2017/00898; A61B 2017/1205

(57) ABSTRACT

An embolic coil includes a coil body including a wire extending spirally, and a groove is formed on a surface of the wire.

### CROSS-REFERENCES TO RELATED APPLICATIONS

This application is a continuation of International Patent Application No. PCT/JP2022/022855 filed on Jun. 6, 2022, which claims priority to Japanese Patent Application No. 2021-145724 filed on Sep. 7, 2021, the entire content of both of which is incorporated herein by reference.

### TECHNOLOGICAL FIELD

The present invention generally relates to an embolic coil.

### BACKGROUND DISCUSSION

One example of a known embolization is an embolization in which a blood vessel, an aneurysm, or the like of a brain or an abdomen is occluded. An example of an embolization device used for an embolization is an embolic coil. It is possible to occlude an inner cavity by densely filling the inner cavity such as a blood vessel or an aneurysm with the embolic coil. Japanese Unexamined Patent Application Publication No. 10-198 (JPH10-198A) discloses an embolic coil of this type.

### SUMMARY

The embolic coil preferably has high flexibility. By using an embolic coil with highly flexibility, it is possible to densely fill the inner cavity such as a blood vessel or an aneurysm with the embolic coil. There is still room for improvement from the viewpoint of flexibility in the embolic coil described in Japanese Unexamined Patent Application Publication No. 10-198.

The embolic coil disclosed here exhibits high flexibility.

According to a first aspect, an embolic coil includes a coil body including a wire extending spirally, and a groove is formed on a surface of the wire.

In one embodiment, the groove extends in a direction intersecting an extending direction of the wire.

In one embodiment, the groove is a spiral groove extending spirally along the extending direction of the wire.

In one embodiment, the groove is an endless groove continuous over an entire region in a circumferential direction of the wire, and a plurality of the endless grooves are disposed at intervals along the extending direction of the wire.

In one embodiment, the groove is an end groove located only in a portion in a circumferential direction of the wire, and a plurality of the end grooves are disposed at intervals along the extending direction of the wire.

In one embodiment, the end groove is formed at least at a position exposed on an outer peripheral surface side of the coil body on a surface of the wire.

In one embodiment, the groove extends in a direction orthogonal to the extending direction of the wire or a coil axis direction of the coil body.

In one embodiment, the groove is formed at substantially equal intervals along the extending direction of the wire and over the entire region in the extending direction of the wire.

In one embodiment, a distal end portion of the coil body includes flexible portions formed of portions of the wire where the grooves are formed, and the flexible portions are disposed at positions facing each other in a coil radial direction of the coil body and at different positions in a coil axis direction of the coil body.

In one embodiment, a swellable body configured to swell by coming into contact with a body fluid is further provided, and the swellable body covers an outside of the coil body in the coil radial direction or is accommodated inside the coil body in the coil radial direction.

By virtue of the construction of the embolic coil, it is possible to provide an embolic coil with high flexibility.

According to another aspect, an embolic coil comprises a coil body constituted by an elongated coil portion that is helically coiled so that the coil body is a helical coil body that has an inner surface surrounding an interior of the helical coil body; the elongated coil portion being constituted by an elongated wire that is spirally wound around a coil axis, wherein the elongated wire possesses an outer surface having a 360° circumferential extent and extending along a longitudinal extent of the elongated wire. At least one groove is provided in the outer surface of the elongated wire, the at least one groove extending around at least a portion of the 360° circumferential extent of the outer surface of the elongated wire.

In accordance with a further aspect, a method comprises moving a catheter towards an indwelling position in an inner cavity of a patient's living body while an embolic coil is positioned inside the catheter. The embolic coil comprises a coil body in which a wire extends spirally, with the wire possessing an outer surface, and the outer surface of the wire being provided with at least one groove. The method additionally involves moving the embolic coil from inside the catheter to a position outside the catheter to deliver the embolic coil to the indwelling position in the inner cavity. The moving of the embolic coil from inside the catheter to the position outside the catheter causes the coil body to form loops in the inner cavity.

### BRIEF DESCRIPTION OF THE DRAWINGS

FIG. 1 is a diagram showing a use example of an embolic coil according to one embodiment disclosed by way of example here.

FIG. 2 is a diagram showing the use example of the embolic coil according to the embodiment.

FIG. 3 is a diagram showing the use example of the embolic coil according to the embodiment.

FIG. 4 is a diagram showing an outer shape of the embolic coil shown in FIGS. 1 to 3 in a natural state.

FIG. 5 is an enlarged view of a part of a coil body of the embolic coil shown in FIG. 1.

FIG. 6 is an enlarged view of a wire forming a coil portion of the coil body shown in FIG. 5.

FIG. 7 is a diagram showing a modification of grooves formed in a surface of the wire.

FIG. 8A is a diagram showing another modification of the grooves formed in the surface of the wire.

FIG. 8B is an enlarged view of the wire in which the grooves shown in FIG. 8A are formed.

FIG. 9 is a diagram showing a modification of a swellable body shown in FIG. 5.

FIG. 10A is a side view of a coil body in which the grooves are not formed over an entire region of the wire in an extending direction.

FIG. 10B is a view showing a formation position of a groove in a coil circumferential direction in the coil body shown in FIG. 10A.

FIG. 11 is a view showing another use example of the embolic coil shown in FIG. 1.

### DETAILED DESCRIPTION

Hereinafter, embodiments of an embolic coil, representing examples of the new embolic coil disclosed here, will be described with reference to the drawings. In the drawings, the same components are denoted by the same reference numerals. In the present specification, a coil axis direction of a coil body of the embolic coil is referred to as a “coil axis direction A”. A coil circumferential direction of the coil body of the embolic coil is referred to as a “coil circumferential direction D”. In addition, a coil radial direction of the coil body of the embolic coil is referred to as a “coil radial direction E”.

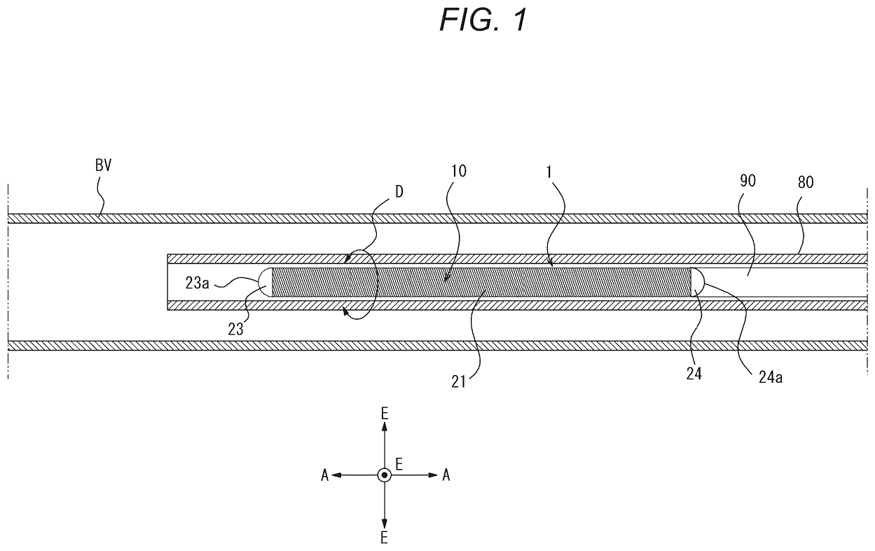

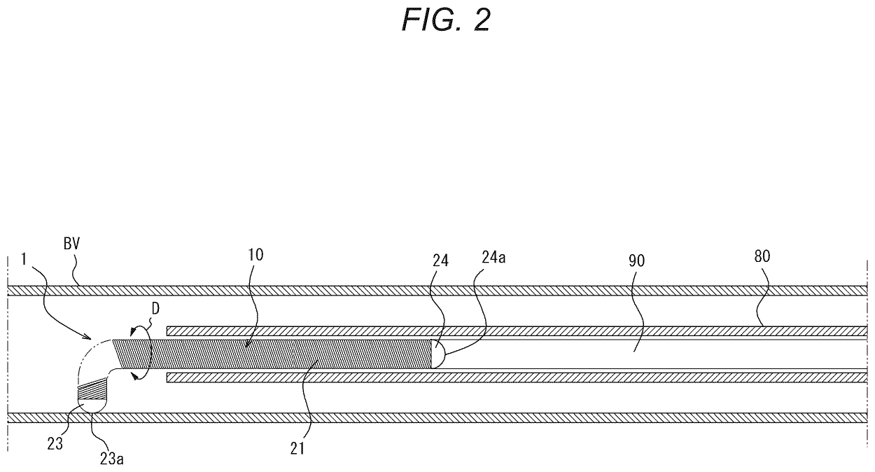

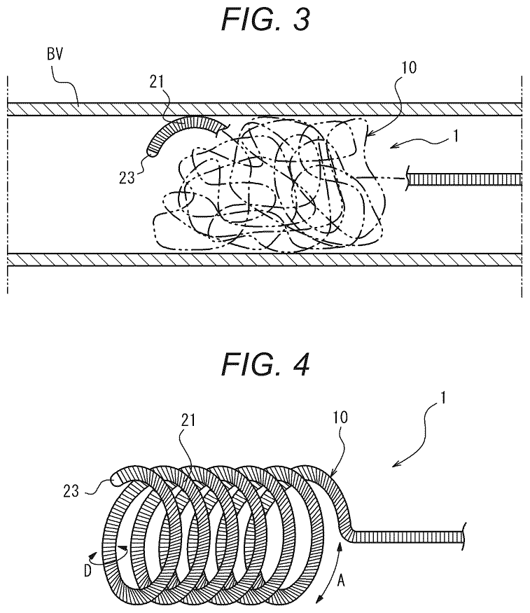

FIGS. 1 to 3 are diagrams showing a use example of an embolic coil 1 as one embodiment of the embolic coil disclosed here. FIG. 1 is a diagram showing a state in which the embolic coil 1 is being delivered from the outside of a patient's living body to an indwelling position in a blood vessel BV. FIG. 2 is a diagram showing a state in which the embolic coil 1 delivered to the indwelling position in the blood vessel BV is being filled in the indwelling position in the blood vessel BV. FIG. 3 shows a state in which the embolic coil 1 is filled in the indwelling position in the blood vessel BV and indwelled.

As shown in FIG. 1, the embolic coil 1 is delivered to the indwelling position in the blood vessel BV through a catheter 80. The indwelling position here means a position to be occluded in the blood vessel BV. Examples of the position to be occluded include, but are not limited to, a position at which blood flow to a vascular malformation or tumor is blocked. The embolic coil 1 may be pressed toward a distal end side of the catheter 80 by an extrusion tool 90 inserted from a proximal end of the catheter 80. Accordingly, the embolic coil 1 can move in the catheter 80 from a proximal side to a distal side of the catheter 80.

As shown in FIG. 2, at the indwelling position in the blood vessel BV, the embolic coil 1 is pushed or discharged into the blood vessel BV from the distal end of the catheter 80 by the extrusion tool 90. Then, as shown in FIG. 3, the embolic coil 1 s filled at the indwelling position in the blood vessel BV. Thereafter, a connection of a connection portion of the embolic coil 1 (not shown) to be connected to the extrusion tool 90 is released by a predetermined mechanism, and the embolic coil 1 is indwelled (i.e., remains in the blood vessel BV).

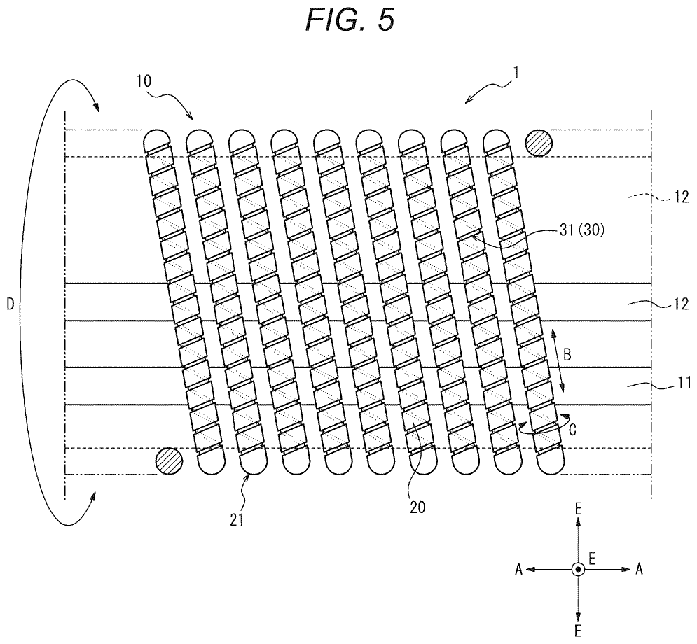

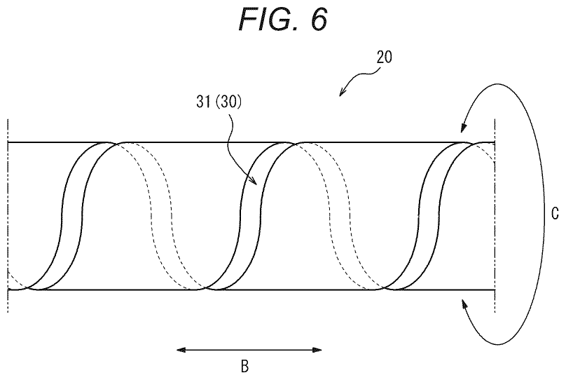

FIG. 4 is a diagram showing an outer shape of the embolic coil according to the embodiment in a natural state. The “natural state” means a no-load state in which no external force acts on or is applied to the embolic coil. As shown in FIG. 4, a coil body 10 of the embolic coil 1 according to the embodiment is formed in advance into a spiral shape (shape memory). That is, the coil body 10 of the embolic coil 1 according to the embodiment is formed so that an elongated coil portion 21 formed by spirally extending a wire 20 (see FIGS. 5 and 6) is further spirally extended. Described differently, an elongated wire 20 is spirally wound to form the coil portion 21, which is an elongated coil portion 21. The elongated coil portion 21 of the coil body 10 is then helically coiled to result in an embolic coil 1 which is helically coiled such as shown in FIG. 4. Hereinafter, for convenience of description, a spiral formed by the wire 20 (see FIGS. 5 and 6) is referred to as a “primary spiral”, and a spiral formed by the coil portion 21 is referred to as a “secondary spiral”. As shown in FIG. 4, the coil portion 21 is helically coiled to form the coil body 10 having an inner surface surrounding an interior of the helical coil body.

In the embodiment, as shown in FIGS. 1 and 2, the coil body 10 is accommodated in the catheter 80 in a state where the secondary spiral of the coil portion 21 is extended linearly. That is, the coil body 10 according to the embodiment is accommodated in the catheter 80 in a state where the coil portion 21 forming the secondary spiral in the natural state is more linearly corrected (straightened) by an inner wall of the catheter 80. As shown in FIGS. 2 and 4, the coil portion 21 of the coil body 10 is pushed out from the distal end of the catheter 80, so that the coil portion 21 returns to the spiral shape from a linear shape by a restoring force, and forms the secondary spiral.

A coil outer diameter in the natural state of the secondary spiral formed by the coil portion 21 of the coil body 10 according to the embodiment is larger than an inner diameter of the blood vessel BV. When the coil body 10 protrudes from the catheter 80 and a distal end of the coil body 10 forms a loop, as shown in FIG. 3, the coil body 10 forms loops in various directions to create a lump that occludes the inside of the blood vessel BV. The lump of the coil body 10 is in close contact with the inner wall of the blood vessel BV. That is, the coil body 10 is indwelled in the blood vessel BV without being pushed away by a blood flow.

For convenience of explanation, FIG. 3 shows a state in which one embolic coil 1 is indwelled at the indwelling position of the blood vessel BV, but a plurality of embolic coils 1 may be densely filled in order to occlude the indwelling position of the blood vessel BV. For example, an upstream side (right side in FIG. 3) of the lump of the coil body 10 of the embolic coil 1 shown in FIG. 3 may be filled with another embolic coil 1.

Further, as shown in FIG. 4, the coil body 10 of the embolic coil 1 is formed in advance, so that the coil portion 21 forms the secondary spiral, but the secondary shape is not limited to a spiral shape. The coil portion 21 may be formed in a secondary shape different from the secondary spiral, for example, a spherical shape or a five-wheel shape, according to an inner cavity shape of the indwelling position.

In FIGS. 1 to 3, a tubular portion of the blood vessel BV is exemplified and described as the indwelling position of the embolic coil 1, but the indwelling position is not limited thereto. The indwelling position of the embolic coil 1 may be, for example, a space inside an aneurysm (see FIG. 11).

Next, with reference to FIGS. 1 to 6, the embolic coil 1 will be described in detail. FIG. 5 is an enlarged view of a part of the coil body 10 of the embolic coil 1. FIG. 6 is an enlarged view of the wire 20 forming the coil portion 21 of the coil body 10 shown in FIG. 5.

As shown in FIG. 1 and the like, the embolic coil 1 includes the coil body 10. The coil body 10 includes the wire 20 extending spirally. More specifically, the coil body 10 according to the embodiment includes the coil portion 21 formed by spirally extending the wire 20. As described above, the coil portion 21 according to the embodiment is formed in advance so as to form the secondary spiral in the natural state (see FIG. 4).

Examples of a material forming the wire 20 include platinum, gold, palladium, tungsten, tantalum, cobalt, rhodium, titanium, alloys thereof, stainless steel, nickel alloys, molybdenum alloys, and Ni—Ti alloys (nitinol).

Further, as shown in FIG. 1, the coil body 10 according to the embodiment includes a first head portion 23 connected to a distal end of the coil portion 21 formed of the wire 20, and a second head portion 24 connected to a proximal end of the coil portion 21. As shown in FIG. 1, the first head portion 23 and the second head portion 24 according to the embodiment have substantially the same diameter (outer diameter) as the coil outer diameter of the coil portion 21, but sizes thereof are not particularly limited. In addition, although a distal surface 23a of the first head portion 23 and a proximal surface 24a of the second head portion 24 according to the embodiment are formed of convex curved surfaces, shapes thereof are not particularly limited.

As shown in FIG. 5, the embolic coil 1 according to the embodiment further includes an elongation resistor 11 and a swellable body 12.

The elongation resistor 11 may be formed of, for example, a resin or metal wire member or a tubular member. The elongation resistor 11 according to the embodiment extends along a coil axis direction of the coil portion 21 (the same direction as the coil axis direction A of the coil body 10) inside the coil portion 21 of the coil body 10. Both ends of the elongation resistor 11 may be fixed to, for example, the first head portion 23 and the second head portion 24 of the coil body 10. By providing such an elongation resistor 11, excessive elongation of the coil portion 21 of the coil body 10 is prevented.

The swellable body 12 is formed of a high molecular material which is swollen by moisture in blood when the material comes into contact with blood as a body fluid. The swellable body 12 may be formed of, for example, a hydro gel formed in a linear or tubular shape. The hydro gel has a polymer chain crosslinked in a three-dimensional network shape. In a dry state, the polymer chains are in an entangled state. When water molecule diffuses into the polymer chains, the polymer chains are loosened, and the network structure includes the water molecule and swells. As the hydro gel, for example, polyacrylic acid, polymethacrylic acid, polyacrylamide, polyhydroxyethyl methacrylate and derivatives thereof, crosslinked polymers of polyols such as polyvinyl alcohol, polyvinyl pyrrolidone and polyethylene glycol, or polysaccharide-based hydro gels can be used.

As shown in FIG. 5, the swellable body 12 according to the embodiment extends along the coil axis direction A inside the coil portion 21 of the coil body 10. The swellable body 12 according to the embodiment swells so as to occlude an internal space of the coil portion 21 of the coil body 10 by moisture in the blood. In

FIG. 5, the swellable body 12 in a dry state is indicated by a solid line, and the swellable body 12 in a swollen state is indicated by a broken line.

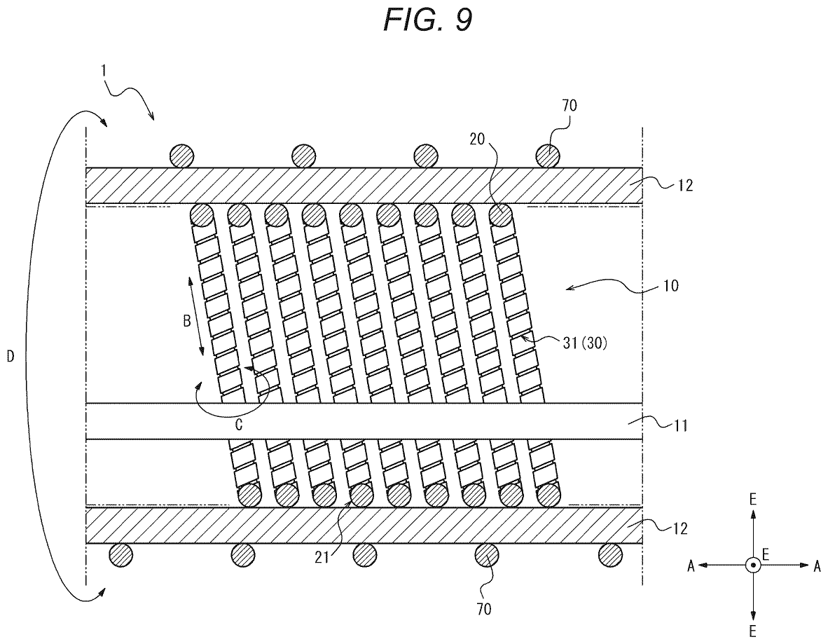

As shown in FIG. 5, the swellable body 12 according to the embodiment is accommodated inside the coil body 10 in the coil radial direction E, but is not limited to this configuration. As shown in FIG. 9, the swellable body 12 may cover the outside of the coil body 10 in the coil radial direction E. That is, the swellable body 12 shown in FIG. 9 is formed in a tubular shape covering a periphery of the coil body 10. FIG. 9 shows a state in which the swellable body 12 is dried. As shown in FIG. 9, a pressing coil 70 is preferably disposed around the swellable body 12. By providing the pressing coil 70, a position of the swellable body 12 can be maintained around the coil body 10. That is, it is possible to prevent separation of the swellable body 12 from the coil body 10. The swellable body 12 can swell so as to protrude outward in the coil radial direction E from a gap of the pressing coil 70. That is, the swellable body 12 can swell so that the swollen body 12 protrudes outward in the coil radial direction E at locations corresponding to the gaps between adjacent windings of the pressing coil 70.

As described above, the embolic coil 1 according to the embodiment includes the coil body 10, the elongation resistor 11, and the swellable body 12, but it is sufficient that the coil body 10 including the wire 20 forming at least a temporary spiral is provided, and other configurations are not particularly limited.

Next, the wire 20 will be described in detail. As shown in FIGS. 5 and 6, a groove 30 is formed on the surface (outer surface) of the wire 20. By forming the groove 30 on the surface of the wire 20, bending rigidity of the coil body 10 including the wire 20 extending spirally can be reduced, and flexibility of the coil body 10 can be improved.

As described above, by improving the flexibility of the coil body 10, the coil body 10 having a large coil outer diameter (for example, 0.035 inch or the like), which is used when a relatively thick blood vessel such as an abdominal artery or a deep vein is embolized, may also be used when, for example, small peripheral blood vessels are embolized. In this case, it is not necessary to use the coil body 10 having a different coil outer diameter according to a thickness of the blood vessel.

The groove 30 preferably extends in a direction intersecting the extending direction B of the wire 20. Examples of the groove 30 include a spiral groove 31 according to the embodiment spirally extending along the extending direction B of the wire 20. The spiral groove 31 means a groove extending in the direction intersecting the extending direction B of the wire 20 over at least one round (360° or more) in a circumferential direction C of the wire 20. The spirally extending or helically extending groove 31 includes axially adjacent groove portions that are disposed at intervals or axially spaced apart from one another.

A pitch width of the spiral groove 31 in the extending direction B of the wire 20 is not particularly limited. The pitch width can be appropriately set according to desired flexibility required for the coil body 10.

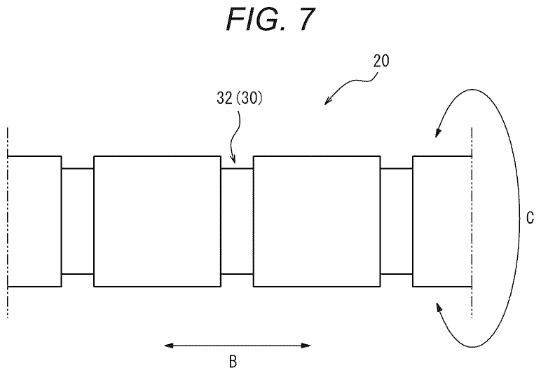

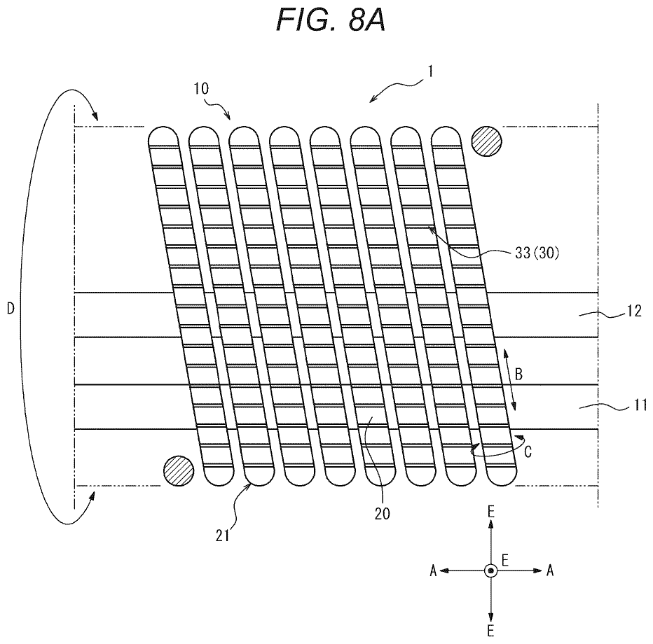

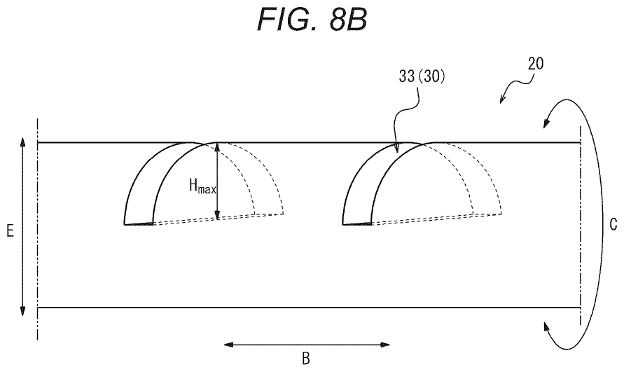

The groove 30 extending in the direction intersecting the extending direction B of the wire 20 is not limited to the spiral groove 31 according to the embodiment. The groove 30 extending in the direction intersecting the extending direction B of the wire 20 may be, for example, an endless groove (see FIG. 7) or an end groove (see FIGS. 8A and 8B).

FIG. 7 shows a modification of the groove 30 formed on the surface of the wire 20. The groove 30 shown in FIG. 7 is an endless groove 32 extending over the entire region of the wire 20 in the circumferential direction C. The endless groove 32 is formed in an annular shape along the circumferential direction C of the wire 20. As shown in FIG. 7, a plurality of endless grooves 32 are disposed at intervals along the extending direction B of the wire 20 (axial extent of the wire). The endless grooves 32 define axially adjacent groove portions that are disposed at intervals or axially spaced apart from one another. A distance between two adjacent endless grooves 32 in the extending direction B (axial direction) is not particularly limited. The distance may be appropriately set according to the desired flexibility required for the coil body 10.

In addition, the endless groove 32 shown in FIG. 7 extends in a direction orthogonal to the extending direction B of the wire 20, but is not limited to this configuration. The endless groove 32 may extend in a direction inclined at less than 90° with respect to the extending direction B of the wire 20.

FIGS. 8A and 8B show another modification of the groove 30 formed on the surface of the wire 20. FIG. 8A is an enlarged view of a part of the coil body. FIG. 8B is an enlarged view of the wire 20 forming the coil portion 21 of the coil body 10 shown in FIG. 8A. In FIG. 8B, an upper side with respect to the wire 20 is the outside in the coil radial direction E, and a lower side with respect to the wire 20 is the inside in the coil radial direction E. The grooves 30 shown in FIGS. 8A and 8B are end grooves 33 that have a limited circumferential extent and terminate at two ends or opposite ends. As shown in FIG. 8B, the end grooves 33 are located only in a part of the wire 20 in the circumferential direction C. In other words, the end groove 33 extends in the direction intersecting the extending direction B of the wire 20 over less than one round (less than 360°) in the circumferential direction C of the wire 20. As shown in FIGS. 8A and 8B, a plurality of end grooves 33 are disposed at intervals along the extending direction B of the wire 20. The end grooves 33 define axially adjacent groove portions that are disposed at intervals or axially spaced apart from one another. A distance (axial distance) between two adjacent end grooves 33 in the extending direction B is not particularly limited. The distance may be appropriately set according to the desired flexibility required for the coil body 10. A maximum depth Hₘₐₓ of the end groove 33 shown in FIG. 8B is also not particularly limited. However, from the viewpoint of preventing plastic deformation of the wire 20 at positions of the end grooves 33, the maximum depth Hₘₐₓ is preferably ⅔ or less, more preferably ½ or less, and particularly preferably ⅓ or less of a maximum diameter of the wire 20.

In addition, as shown in FIG. 8A, the end groove 33 is formed at least at a position exposed on an outer peripheral surface side of the coil body 10 on the surface of the wire 20. In other words, the end groove 33 is formed at least at a position exposed on the outer peripheral surface side of the coil portion 21 of the coil body 10 on the surface of the wire 20. By providing the end groove 33 at such a position, the end groove 33 can be easily formed from the outside of the coil body 10 even after the wire 20 is spirally formed (spirally wound) to form the coil portion 21. However, the position of the end groove 33 is not limited to the positions shown in FIGS. 8A and 8B, and the end groove 33 may be formed at a position exposed on an inner peripheral surface side of the coil body 10 on the surface of the wire 20.

Further, the end grooves 33 shown in FIGS. 8A and 8B extend in the direction intersecting the extending direction B of the wire 20. The end groove 33 extending in the direction intersecting the extending direction B of the wire 20 may extend, for example, in the direction orthogonal to the extending direction B of the wire 20 or in the coil axis direction A of the coil body 10. The end groove 33 may extend in a direction inclined at less than 90° with respect to the extending direction B of the wire 20. However, as shown in FIGS. 8A and 8B, the end groove 33 formed at a position exposed on the outer peripheral surface side of the coil body 10 preferably extends along the coil axis direction A of the coil body 10. In this manner, after the wire 20 is spirally formed to form the coil portion 21, for example, by being linearly irradiated with a laser cutter or the like along the coil axis direction A, the plurality of end grooves 33 disposed along the coil axis direction A can be formed at a time. That is, the plurality of end grooves 33 can be easily formed.

As in the embodiment, when the embolic coil 1 includes the swellable body 12, a part of the swellable body 12 which is swollen enters the above groove 30. Therefore, it is possible to prevent the swellable body 12 from slipping and being displaced with respect to the coil body 10. The groove 30 is preferably the spiral groove 31 (see FIGS. 5 and 6) or the endless groove 32 (see FIG. 7) provided over the entire region in the circumferential direction C of the wire 20. By using the spiral groove 31 or the endless groove 32 as the groove 30, as compared with the configuration in which the end grooves 33 (see FIGS. 8A and 8B) are used as the groove 30, an extending length of the groove 30 is increased, a portion of the swellable body 12 to be caught by the groove 30 is increased, and thus, the displacement of the swellable body 12 with respect to the coil body 10 can be further prevented.

Further, the groove 30 preferably has a component extending in a direction inclined with respect to the coil axis direction A. In this manner, it is possible to prevent the swellable body 12 from slipping in the coil axis direction A with respect to the coil body 10. Further, the groove 30 is particularly preferably the spiral groove 31. The spiral groove 31 includes not only a component extending in a direction inclined with respect to the coil axis direction A but also components extending in various directions. Therefore, not only in the coil axis direction A but also in other directions, the swellable body 12 can be more reliably prevented from slipping with respect to the coil body 10. The spiral groove 31 can be easily formed by linearly irradiating the wire 20 in the extending direction B of the wire 20 with, for example, a laser cutter while rotating the wire 20 in the circumferential direction with respect to the wire 20 before forming the primary spiral.

Next, a position where the groove 30 is formed in the coil axis direction A and the coil circumferential direction D of the coil body 10 will be described.

From the viewpoint of improving the flexibility of the entire coil body 10, the grooves 30 are preferably formed at substantially equal intervals along the extending direction B of the wire 20 and over the entire region in the extending direction B of the wire 20. In this manner, the grooves 30 are disposed at substantially equal intervals over the entire region of the coil body 10 in each of the coil axis direction A and the coil circumferential direction D. The above-described “grooves 30 are formed at substantially equal intervals along the extending direction B of the wire 20” means that, when the grooves 30 are the spiral grooves 31, the grooves 30 being formed at substantially equal pitch widths in the extending direction B of the wire 20 and/or the distance between two adjacent spiral grooves 31 of the plurality of spiral grooves 31 disposed in the extending direction B of the wire 20 being substantially equal is satisfied.

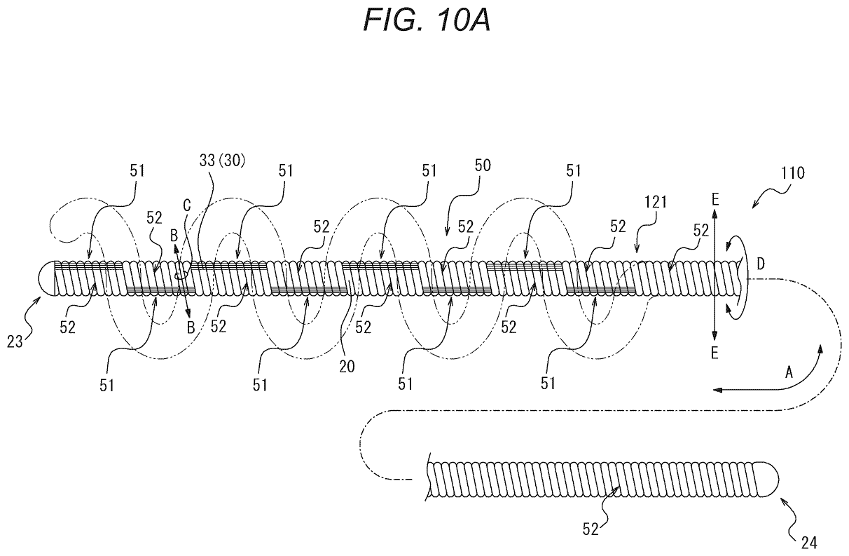

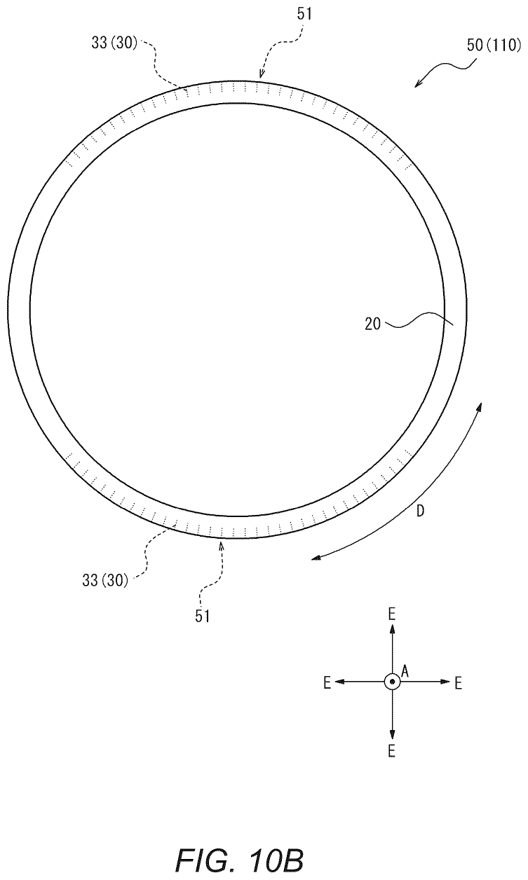

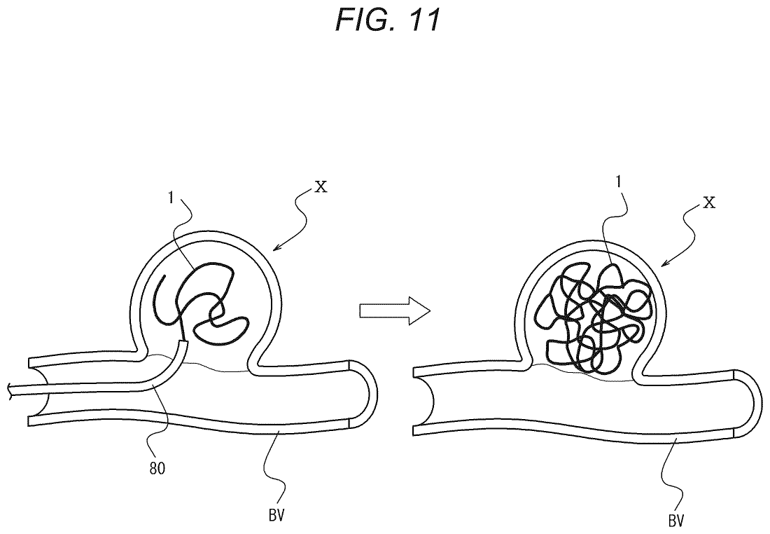

However, the grooves 30 may not be formed at substantially equal intervals along the extending direction B of the wire 20 and over the entire region in the extending direction B of the wire 20. FIGS. 10A and 10B are diagrams showing an example of a coil body 110 in which the groove 30 is not formed over the entire region in the extending direction B of the wire 20. FIG. 10A is a side view of the coil body 110 as viewed from a direction orthogonal to the coil axis direction A of the coil body 110. FIG. 10B is a diagram showing a formation position of the groove 30 in the coil circumferential direction D. In FIG. 10B, the positions of the plurality of grooves 30 densely disposed in the coil circumferential direction D are indicated by broken lines. The coil body 110 shown in FIGS. 10A and 10B has a different length in the coil axis direction A as compared with the coil body 10 shown in FIGS. 1 to 4, but a length of the coil bodies 10, 110 in the coil axis direction A can be changed as appropriate, and the lengths are not particularly limited. Further, in FIGS. 10A and 10B, the end groove 33 (see FIGS. 8A and 8B) is shown as the groove 30, but the groove 30 may be the spiral groove 31 (see FIGS. 5 and 6) or the endless groove 32 (see FIG. 7), and the shape of each groove 30 is not particularly limited.

As shown in FIG. 10A, in a coil portion 121 of the coil body 110, the grooves 30 are not formed at substantially equal intervals along the extending direction B of the wire 20. As shown in FIG. 10A, a distal end portion 50 of the coil body 110 includes flexible portions 51 formed by portions of the wire 20 where the grooves 30 are formed. The flexible portion 51 means a portion where the grooves 30 are formed more densely as compared with adjacent positions in at least one of the coil axis direction A and the coil circumferential direction D. That is, the flexible portion 51 includes non-flexible portions 52 where the grooves 30 are not formed more densely as compared with the flexible portion 51 or where the grooves 30 are not formed at all in at least one of the coil axis direction A and the coil circumferential direction D. The non-flexible portion 52 has higher bending rigidity as compared with the flexible portion 51. The flexible portions 51 and the non-flexible portions 52 may be defined by, for example, a total length of the grooves 30 per unit area.

In addition, a portion of the coil body 110 shown in FIGS. 10A and 10B on a proximal end side with respect to the distal end portion 50 does not include the flexible portion 51. More specifically, the groove 30 is not formed at all in a portion of the coil body 110 shown in FIG. 10A on the proximal end side in the coil axis direction A with respect to the distal end portion 50. In other word, the portion of the coil body 110 shown in FIG. 10A on the proximal end side in the coil axis direction A with respect to the distal end portion 50 is only formed of the non-flexible portion 52.

As shown in FIG. 10A, in the distal end portion 50 of the coil body 110, the plurality of flexible portions 51 are disposed at different positions in the coil axis direction A of the coil body 110. As shown in FIG. 10B, in the distal end portion 50 of the coil body 110, the plurality of flexible portions 51 are disposed at positions facing each other in the coil radial direction E of the coil body 110. That is, in the distal end portion 50 of the coil body 110, the flexible portions 51 are disposed at diametrically opposite positions of the coil body 110 as shown in FIG. 10A. By disposing the flexible portion 51 in the distal end portion 50 of the coil body 110 as described above, the distal end portion 50 of the coil body 110 is easily deformed into a wave shape as indicated by a two-dot chain line in FIG. 10A. Therefore, kickback when the coil body 110 is filled into (positioned in) the blood vessel BV (see FIG. 1 and the like) can be prevented. The kickback of the coil body 110 is a phenomenon in which the distal end of the coil body 110 abuts against an inner wall of the blood vessel BV, and the coil body 110 moves to return into the catheter 80 (see FIGS. 1 and 2) by receiving a reaction force of the inner wall of the blood vessel BV. When the distal end of the coil body 110 abuts against the inner wall of the blood vessel BV, the distal end portion 50 of the coil body 110 is deformed in the wave shape, and thus the above-described kickback can be prevented.

In FIGS. 10A and 10B, in the distal end portion 50 of the coil body 110, the plurality of flexible portions 51 are disposed only at different positions in the coil axis direction A and positions facing each other in the coil radial direction E, but the invention is not limited to this configuration. In order to easily deform the distal end portion 50 of the coil body 110 into the wave shape (see the two-dot chain line in FIG. 10A), another flexible portion 51 may be disposed in addition to the flexible portion 51 disposed at the above-described position.

Another usage example of the embolic coil 1 will be described with reference to FIG. 11. Although FIGS. 1 to 3 show an example in which the embolic coil 1 is filled into the tubular blood vessel BV (i.e., the tubular blood vessel BV is filled with the embolic coil 1), the embolic coil 1 may be filled into an aneurysm X as shown in FIG. 11 (i.e., an aneurysm X may be filled with the embolic coil 1). As shown in FIG. 11, the embolic coil 1 is filled in the aneurysm X, while the embolic coil 1 is intertwined so as to fill a gap or space/volume within the aneurysm X. Then, the aneurysm X is occluded by being densely filled with the embolic coil 1.

The detailed description above describes embodiments of an embolic coil representing examples of the new embolic coil disclosed here. The invention is not limited, however, to the precise embodiments and variations described. Various changes, modifications and equivalents can be effected by one skilled in the art without departing from the spirit and scope of the invention as defined in the accompanying claims. It is expressly intended that all such changes, modifications and equivalents that fall within the scope of the claims are embraced by the claims.

What is claimed is:

1. An embolic coil comprising:
a coil body including a wire extending spirally, wherein
a groove is formed on a surface of the wire.

2. The embolic coil according to claim 1, wherein the groove extends in a direction intersecting an extending direction of the wire.

3. The embolic coil according to claim 2, wherein the groove is a spiral groove extending spirally along the extending direction of the wire.

4. The embolic coil according to claim 2, wherein the groove is one of a plurality of endless grooves, each of the endless grooves being continuous over a circumferential direction of the wire, and the plurality of endless grooves are spaced apart from one another along the extending direction of the wire.

5. The embolic coil according to claim 2, wherein the groove is one of plural end grooves, each end groove extending less than 360° around a circumference of the wire so that the end groove extends between and terminates at two ends, the plural end grooves being disposed at intervals along the extending direction of the wire.

6. The embolic coil according to claim 5, wherein the coil body includes a radially outwardly facing outer peripheral surface side and a radially inwardly facing inner peripheral surface side, each of the plural end grooves being located on the wire such that the plural end grooves are exposed on the outer peripheral surface side of the coil body.

7. The embolic coil according to claim 4, wherein each of the end grooves extends in a direction orthogonal to the extending direction of the wire or a coil axis direction of the coil body.

8. The embolic coil according to claim 1, wherein the wire extends spirally in an extending direction to form the coil body, the groove including axially adjacent groove portions positioned at substantially equal intervals along the extending direction of the wire and over an entire region of the wire in the extending direction of the wire.

9. The embolic coil according to claim 1, wherein the groove includes plural grooves, a distal end portion of the coil body including flexible portions at portions of the wire where the grooves are formed, and
the flexible portions are disposed at positions facing each other in a coil radial direction of the coil body and at different positions in a coil axis direction of the coil body.

10. The embolic coil according to claim 1, further comprising:
a swellable body configured to swell by coming into contact with a body fluid, wherein
the swellable body covers an outside of the coil body in the coil radial direction or is accommodated inside the coil body in the coil radial direction.

11. An embolic coil comprising:
a coil body constituted by an elongated coil portion that is helically coiled so that the coil body is a helical coil body that has an inner surface surrounding an interior of the helical coil body;
the elongated coil portion being constituted by an elongated wire that is spirally wound around a coil axis;
the elongated wire possessing an outer surface having a 360° circumferential extent and extending along a longitudinal extent of the elongated wire; and
at least one groove provided in the outer surface of the elongated wire, the at least one groove extending around at least a portion of the 360° circumferential extent of the outer surface of the elongated wire.

12. The embolic coil according to claim 11, wherein the elongated wire extends spirally in an extending direction to form the coil portion, the groove extending in a direction intersecting the extending direction of the elongated wire.

13. The embolic coil according to claim 11, wherein the groove is a spiral groove that extends spirally along the outer surface of the elongated wire so that the groove extends more than 360° around the outer surface of the elongated wire.

14. The embolic coil according to claim 11, wherein the groove is one of a plural endless grooves that are separate from one another and axially spaced apart from one another along at least a portion of the longitudinal extent of the elongated wire.

15. The embolic coil according to claim 11, wherein the at least one groove extends along at least a portion of the longitudinal extent of the elongated wire, the portion of the longitudinal extent of the elongated wire being a distal portion of the elongated wire.

16. The embolic coil according to claim 11, wherein the elongated wire extends spirally in an extending direction to form the coil portion, the groove being one of plural grooves that are spaced apart from one another along the extending direction of the wire.

17. The embolic coil according to claim 16, wherein each of the grooves extends around the circumferential extent of the wire less than 360°, each of the grooves terminating at opposite ends that are spaced apart from one another.

18. The embolic coil according to claim 11, wherein the groove is one of plural grooves, the coil body including a radially outwardly facing outer peripheral surface side and a radially inwardly facing inner peripheral surface side, each of the plural grooves being located on the wire such that the plural grooves are on the outer peripheral surface side of the coil body.

19. A method comprising:
moving a catheter towards an indwelling position in an inner cavity of a patient's living body while an embolic coil is positioned inside the catheter, the embolic coil comprising a coil body in which a wire extends spirally, the wire possessing an outer surface, the outer surface of the wire being provided with at least one groove;
moving the embolic coil from inside the catheter to a position outside the catheter to deliver the embolic coil to the indwelling position in the inner cavity; and
the moving of the embolic coil from inside the catheter to the position outside the catheter causing the coil body to form loops in the inner cavity.

20. The method according to claim 19, wherein the inner cavity is a blood vessel in the living body or an aneurysm in the living body.
